(19) United States
(12) Patent Application Publication
(10) Pub. No.: US 2025/0027129 A1
(43) Pub. Date: Jan. 23, 2025

(54) DNA SYNTHESIS USING MAGNETIC NANOPARTICLES

(71) Applicant: Seagate Technology LLC, Fremont, CA (US)

(72) Inventors: Riyan Alex MENDONSA, Edina, MN (US); Gemma Roselle MENDONSA, Edina, MN (US); Thomas Young CHANG, Menlo Park, CA (US)

(21) Appl. No.: 18/478,642

(22) Filed: Sep. 29, 2023

Related U.S. Application Data
(60) Provisional application No. 63/377,995, filed on Sep. 30, 2022.

(51) Int. Cl. C12P 19/34; B01J 19/00

(52) U.S. Cl. CPC C12P 19/34; B01J 19/0046; B01J 2219/00454; B01J 2219/00461; B01J 2219/00722; B01J 2219/0807; B01J 2219/12

(57) ABSTRACT

A method for serial and contemporaneous synthesis of disparate deoxy-ribonucleic acid (DNA) strands in an array of wells defined within a substrate. Each well in the array of wells contains a precursor nucleotide chain. A first subset of wells of the array of wells is designated not to receive a nucleotide of a specified nucleobase type and the first subset of wells is closed. A solution of a binding reaction enzyme and nucleotides of the specified nucleobase type bound with a corresponding chemical blocker is flowed over the array of wells. Nucleotides of the specified nucleobase type are received in each of a second subset of wells of the array of wells that are open and designated to receive the nucleotide of the specified nucleobase type. Received nucleotides of the specified nucleobase type are bound with assistance of the binding reaction enzyme to corresponding precursor nucleotide chains in the second subset of wells.

### CROSS-REFERENCE TO RELATED APPLICATIONS

This application is a non-provisional application based on and claims benefit of priority to U.S. provisional patent application No. 63/377,995 filed on Sep. 30, 2022, and entitled DNA SYNTHESIS USING MAGNETIC NANOPARTICLES, which is incorporated herein by reference in its entireties.

### BACKGROUND

Demand for increased capacity in computer data storage, as well as increased speed writing to and reading from that data storage, consistently drives advances in the data storage industry. Achieving cost reduction in data storage devices is also a driver for improvement. Deoxy-ribonucleic acid (DNA) and ribonucleic acid (RNA) provide the foundation for an emerging technology for data storage. (For the purposes of this document, all further references to DNA should be understood to refer to both DNA and RNA.) A DNA strand can be synthesized to store data digitally by ordered combinations of the nucleotides, with each nucleotide, ordered combinations of nucleotides, or transitions between nucleotides, functioning as digital bits. DNA nucleotides are nanoscale in size and DNA strands of hundreds of millions of nucleotides can fold up upon themselves to fit in an extremely small volume, e.g., within the nucleus of a human cell. Thus, the data storage capacity of synthesized DNA is huge and the physical storage space required is extremely small. DNA is also easily replicable through chemical processes, allowing copies of encoded DNA strands to be easily made. Further, techniques have been developed to decode or “read” the data stored in synthesized DNA, for example, by measurement of electrical resistance of each nucleotide as the DNA strand passes through a pore configured with a voltage differential.

The information included in this Background section of the specification, including any references cited herein and any description or discussion thereof, is included for technical reference purposes only and is not to be regarded subject matter by which the scope of the invention as defined in the claims is to be bound.

### SUMMARY

In an example implementation, a method for serial and contemporaneous synthesis of disparate deoxy-ribonucleic acid (DNA) or ribonucleic acid (RNA) strands is performed using an array of wells defined within a substrate. Initially, a first subset of wells of the array of wells defined within the substrate is closed. Each well in the array of wells contains a precursor nucleotide chain, and the first subset of wells is designated as proscribed from receiving a nucleotide of a specified nucleobase type for a cycle of the method. A solution of a binding reaction enzyme and nucleotides of the specified nucleobase type bound with a corresponding chemical blocker are flowed over the array of wells. Nucleotides of the specified nucleobase type are received in each of a second subset of wells of the array of wells that are open and designated to receive the nucleotide of the specified nucleobase type. Binding received nucleotides of the specified nucleobase type are bound, with assistance of the binding reaction enzyme, to corresponding precursor nucleotide chains in the second subset of wells.

In another example implementation, a control system for serial and contemporaneous synthesis of disparate deoxy-ribonucleic acid (DNA) or ribonucleic acid (RNA) strands in an array of wells defined within a substrate is provided. The control system includes a controller including a processor and a memory. The memory further includes a control application and a data structure stored thereon. The control application is executable by the processor to designate a first subset of wells in the array of wells defined within the substrate and containing a precursor nucleotide chain as proscribed from receiving a nucleotide of a specified nucleobase type for a synthesis cycle. The designations is based, at least in part, upon sequence instruction data stored in the data structure. The control application is further executable to close the first subset of wells. The control application is additionally executable to flow a solution of a binding reaction enzyme and nucleotides of the specified nucleobase type bound with a corresponding chemical blocker over the array of wells for receipt in each of a second subset of wells of the array of wells that are open as designated by the sequence instruction data to receive the nucleotide of the specified nucleobase type for binding of the received nucleotides of the specified nucleobase type with assistance of the binding reaction enzyme to corresponding precursor nucleotide chains in the second subset of wells.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. A more extensive presentation of features, details, utilities, and advantages of the present invention as defined in the claims is provided in the following written description of various embodiments and implementations and illustrated in the accompanying drawings.

### BRIEF DESCRIPTION OF THE DRAWINGS

The disclosure will be readily understood by the following detailed description in conjunction with the accompanying drawings, wherein like reference numerals designate like structural elements.

It should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

FIG. 1A is a schematic diagram of an example single-stranded DNA molecule including a plurality of nucleobases extending along a backbone chain formed of corresponding backbone monomers.

FIG. 1B is a schematic diagram of an example double-stranded DNA molecule including a plurality of completed nucleotides forming double helix polymer.

FIG. 2 is a schematic diagram of an example DNA synthesis and storage system including a substrate defining a plurality of nanopore wells for storing DNA strands, magnetic nanoparticles (MNPs) for selectively capping the nanopore wells.

FIG. 3 is a flow diagram of an example process for DNA synthesis and storage within nanopore wells defined within a substrate and using MNPs to selectively cover the nanopore wells.

FIG. 4 is a schematic diagram of a first alternate implementation of a nanopore well.

FIG. 5 is a schematic diagram of a second alternate implementation of a nanopore well.

FIG. 6 is a schematic diagram of a third alternate implementation of a nanopore well.

FIG. 7A is a schematic diagram of a first step in an example implementation for retaining MNPs to cover respective nanopore wells.

FIG. 7B is a schematic diagram of a second step the example implementation of FIG. 7A for retaining certain MNPs to cover respective nanopore wells.

FIG. 7C is a schematic diagram of an alternate second step in the example implementation of FIG. 7A for retaining certain MNPs to cover respective nanopore wells.

FIG. 8A is a schematic diagram of an example implementation of a first step of an alternative process for associating MNPs with respective nanopore wells.

FIG. 8B is a schematic diagram of an example implementation of a second step of the alternative process of FIG. 8A for associating MNPs with respective nanopore wells.

FIG. 9 is a schematic diagram of an example DNA synthesis and storage system including a substrate defining a plurality of nanopore wells for storing DNA strands, magnetic nanoparticles (MNPs) for selectively capping the nanopore wells, and a top plate for assisting in removing MNPs to open selective ones of the nanopore wells according to an example implementation disclosed herein.

FIG. 10 is a schematic diagram of an example circuit configuration for attracting and releasing MNPs to cover and uncover nanopore wells.

FIG. 11 is a schematic diagram of an example switching scheme for attracting and releasing MNPs to cover and uncover nanopore wells.

FIG. 12 is a flow diagram of another example process for DNA synthesis and storage within nanopore wells defined within a substrate and using a top plate to assist in selectively covering and covering the nanopore wells.

FIGS. 13A-D are schematic diagrams that together depict an example method for forming energizable nanopore wells within a substrate and the structure of the substrate defining the energizable nanopore wells.

FIG. 14 is a schematic diagram of a fourth alternate implementation of a nanopore well.

FIG. 15 is a schematic diagram of a fifth alternate implementation of a nanopore well.

### DETAILED DESCRIPTION

In general, this disclosure relates to methods for synthesizing deoxyribonucleic acid (DNA) (e.g., for data storage purposes) in parallel or in an array using electromagnetic control of magnetic nanoparticles (MNPs) to open and close wells (e.g., nanopores) in a substrate in which chemical DNA synthesizes by serialized addition of nucleotides occurs. DNA synthesis in an array as disclosed herein allows for very large numbers of DNA strands to be encoded with disparate and discrete data, up to tens or more terabytes of data within a single ship-sized substrate. The disclosure also provides examples of substrate structures and related mechanisms controllable for managing the MNPs to addressable encode the DNA strands in the array.

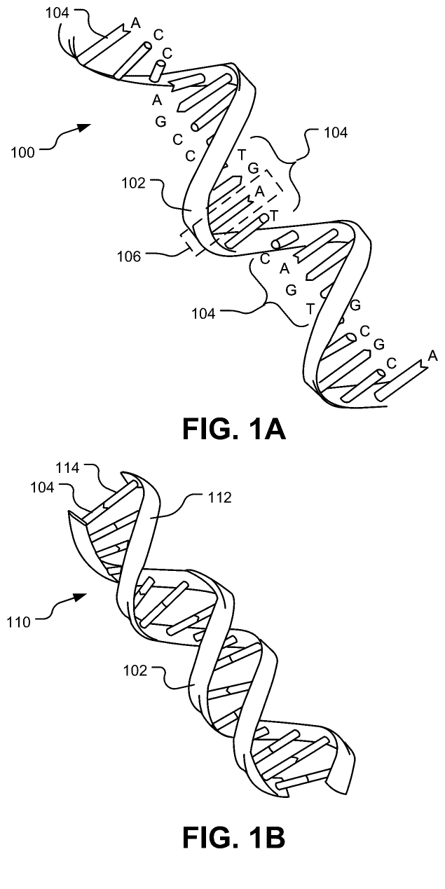

For purposes of general understanding, FIG. 1A depicts a length of single-stranded DNA 100 composed of a backbone chain 102 and a plurality of nucleobases 104 attached along the backbone chain 102 in an apparently random order. The backbone chain is composed of a sugar molecule (2′-deoxyribose) and a phosphate group that link together in alternating pairs. Each sugar molecule attaches to a nucleobase 104. There are four types of nucleobases 104 as indicated in FIG. 1A, wherein the letters adjacent to each nucleobase 104 in FIG. 1A refer to the first letter of the type of nitrogen compound forming the nucleobase: Adenine, Guanine, Cytosine, and Thymine. The combination of a sugar molecule, a phosphate group, and a nucleobase 104 is referred to as a nucleotide 106. FIG. 1B depicts a double-stranded DNA molecule 110 that includes the prior backbone chain 102 and nucleobases 104 of FIG. 1A as well as a conjugate backbone chain 112 and conjugate nucleobases 114. The nucleobases 104 and conjugate nucleobases 114 chemically bond to join the backbone chain and the conjugate backbone chain 112 together as double stranded DNA molecule 110 in the form of a double helix. However, there are limitations on nucleobase bonding: adenine will only bond with thymine (A-T) and cytosine will only bond with guanine (C-G), and vice versa.

DNA strands, both single-sided and double sided, are long, linear polymer chains that may consist of hundreds, thousands, or millions of nucleotides. For example, the human genome is composed of 3.2 billion nucleotides across 24 separate chromosomes (DNA strands). The longest chromosome has approximately 260 million nucleotides and extends about 2 meters in length when unfolded. However, all of these billions of nucleotides in DNA strands fit within the nucleus of each human cell. DNA is a natural storage device; it contains all of the instructions for replicating a living organism, e.g., a human being. It is easy to see, therefore, how synthesized DNA strands with ordered combinations of nucleobases translated to stand for digital data could be leveraged for writing and storing vast amounts of data in an incredible small volume or density. Improving the speed of writing and volume of data written are thus desirable goals.

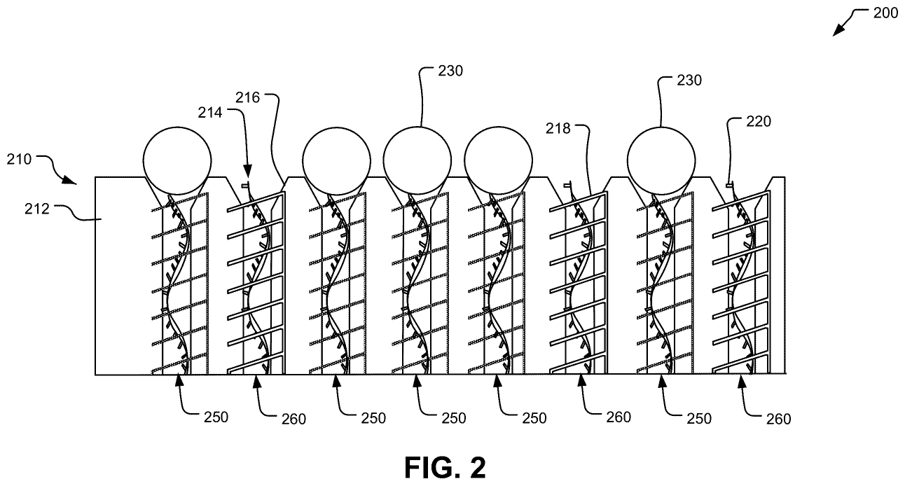

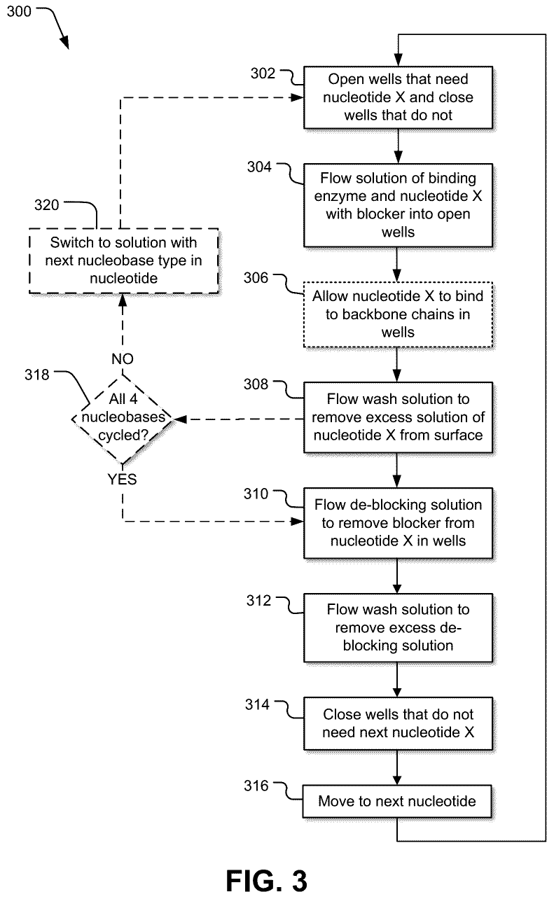

FIG. 2 is a schematic diagram of an example implementation of a DNA synthesis apparatus 200 for use in synthesizing DNA strands, for example, following the method described with respect to FIG. 3. The DNA synthesis apparatus 200 may include a storage substrate 212 populated by a plurality nanopore wells 214 arranged in an array pattern in which individual, single-stranded DNA 220 are formed and stored. The storage substrate 212 is depicted in FIG. 2 in cross section, thus showing a subset of only one row of parallel nanopore wells 214. However, in actuality, the storage substrate 212 would be composed of thousands of parallel rows of nanopore wells 214 with thousands of nanopore wells 214 in each row, resulting in millions of nanopore wells 214 in the storage substrate 212. A top edge of each nanopore well 214 may be designed as a particle seat 216 to receive a respective MNP 230 to cover the nanopore well 214. For example, the top edge of each nanopore well 214 may be beveled or recessed to form the particle seat 216 for receiving an MNP 230. Electromagnetic coils 218 may be formed around all or a portion of each respective nanopore well 214 and used to attract, release, or repel a respective MNP 230 as further described herein.

The storage substrate 212 may be made in a similar manner to computer chip manufacture as further described herein. In general, a silicon wafer base may support a number of interconnect layers within which the nanopore wells 214 may be formed. For example, multiple interconnect layers may be built on the wafer and resist patterns may be used to define and maintain well openings. In another example, holes may be bored into the interconnect layers, e.g., of silica or glass, built on the silicon wafer to form the nanopore wells 214. In other implementations, the nanopore wells 214 may be formed in a layer of aluminum on top of a silicon wafer. The nanopore wells 214 may be on the order of 6 nm to 0.1 μm in diameter and about 25 nm deep. The nanopore wells 214 may be cylindrical or tapered frustoconical forms with down to an 80-degree slope from the top to the bottom beneath the particle seat 216. The silicon wafer may then be diced into chips in a similar manner to processor or memory chips with thousands of nanopore wells 214 formed in each chip. In some implementations, a single chip can be configured with millions of nanopore wells 214 to achieve multiple terabytes of capacity.

The MNPs 230 may be addressable controlled to cover and uncover their respective nanopore wells 214 to allow selective addition of a nucleotide to bind with a backbone chain of the single stranded DNA 220 in open nanopore wells 214. Nanopore wells 214 covered by respective MNPs 230 would not receive the selected nucleotide. Each of the four nucleotide types may be introduced to the storage substrate 212 in succession and cyclically to build disparately coded single-stranded DNA 220. In some implementations, the order of nucleotide deposition and bonding in each nanopore well 214 may be directed and controlled to encode digital data according to any of a number of mapping schemes that translate between ordered adjacent pairs of nucleotides in a single-stranded DNA 220 and digital data forms (e.g., binary, decimal, hexadecimal, etc.).

An example method 300 for synthesizing large quantities of disparately encoded single-stranded DNA in an array using, for example, the DNA synthesis apparatus 200 of FIG. 2 is presented in the flow diagram of FIG. 3. Initially, in an opening operation 302, the nanopore wells containing the DNA strands to which a nucleotide type X (i.e., one of Adenine, Guanine, Cytosine, and Thymine) is to be added may be opened, for example, by deenergizing electromagnetic coils around the nanopore wells to release an MNP capping a respective well. Next, in flowing operation 304, a solution of a binding reaction enzyme and chemically blocked nucleotide type X may be flowed or otherwise introduced over the nanopore wells. Example binding reaction enzymes may include terminal deoxynucleotidyl transferase (TdT), topoisomerase, or other appropriate enzymes. The nucleotides are chemically blocked on the “free end” binding location (for example, with benzoyl, pivaloyl, methoxyethyl, methyl, 2-nitrobenzyl, 3′-O-cyanoethyl, allyl, amine, azidomethyl, or tert-butoxy ethoxy) to prevent chains of the nucleotides from forming in the solution and to limit the binding in open nanopore wells to only one nucleotide per cycle, i.e., to prevent formation of homopolymers. At this point, the binding reaction enzyme will automatically activate and complete binding of nucleotide type X to the backbone chains of the DNA strands in each of the open nanopore wells as indicated in binding operation 306. In some implementations, a chemical tether (e.g., with 1-20 nucleotides) may be placed in each of the nanopore wells to provide an initial base (similar to a packet header) on which to begin building a DNA strand.

Once the process of binding nucleotide type X is complete, the method 300 may continue with a washing operation 308 in which a wash solution is flowed over the DNA storage apparatus to remove excess solution of binding reaction enzyme and nucleotide type X from the surface of the storage substrate. Next, in a de-bocking operation 310 a de-blocking solution, for example, sodium nitrite or trichloroacetic acid, is flowed over the storage substrate to remove the chemical blocker from the end nucleotide on the DNA strand to allow for bonding of the next nucleotide assigned for addition to the DNA strand. Another washing operation 312 may be performed to flow a wash solution over the DNA storage apparatus to remove excessed-blocking solution from the open nanopore wells and the surface of the storage substrate. The method 300 continues in a closing operation 314 by closing open nanopore wells that should not receive the next nucleotide type X′ by energizing respective electromagnetic coils to attract MNPs to cover and close those nanopore wells. The method 300 then cycles to attach the next nucleotide type X′ as indicated in cycling operation 316 by returning to opening operation 302 to open any nanopore wells not already open that are assigned to receive the next nucleotide type X′.

While the method 300 of FIG. 3 represents the core steps for leveraging a DNA storage array as disclosed herein, several variations or optional procedures may also be performed to enhance the process. For example, operations 302-308 could be performed or cycled four times, once for each of the nucleobase types, before proceeding with the de-blocking operation 310. As shown in FIG. 3 in an alternative flow, after the washing operation 308, a query operation 318 may alternately be performed to determine whether nucleotide solutions for each of the four nucleobases have been flowed across the storage substrate. If not, a switching operation 320 may be performed to switch to a solution with a next nucleobase type in the nucleotide and then return to opening operation 302 to open any wells that are assigned to receive the next nucleobase in their DNA strand for encoding. Because a blocking chemical has been attached to the free end of the prior nucleotides, the nanopore wells can remain uncovered and open and the next nucleotide will not bind to the DNA strands in those nanopore wells. The method can cycle in this manner four times until nucleotide solutions with each of the nucleobases have been flowed across the storage substrate in flowing operation 304. When all four nucleotide solutions have been used, the query operation 318 will send the method to de-blocking operation 310, which will operate on all end nucleotides of the DNA strands in all of the nanopore wells in the storage substrate as all of the nanopore wells should be uncovered at this point.

In other example implementations, at the end of the method 300 the completed DNA strands may be chemically or physically cleaved off the chemical tethers for storage or reading. Copies of the DNA strands may be made within the nanotube wells by standard polymerase reactions to provide multiple copies of the data for reading. Additionally, a further solution wash with all nucleotide types may be provided to transform the encoded single-stranded DNA into double-stranded DNA for greater stability in storage. In some implementations, the electromagnetic coils in open nanopore wells could be energized using an AC pulse or at low current levels to heat the solutions and help facilitate enzymatic reactions without attracting the MNPs.

Table A set forth below presents several examples of the amount of data storage achievable in a DNA storage apparatus as described above with respect to FIG. 2 and following an example storage process similar to the method of FIG. 3. It is apparent that with relatively short DNA strands (e.g., 256 nucleotides in each DNA strand synthesized), massive amounts of data (e.g., 12.8 TB-25.6 TB) may be stored on an approximately 3 cm² chip. Additionally, the write speed is much faster than traditional hard disk drive (HDD) data storage devices and is about half the speed of a solid-state drive (SSD). Write speeds are a function of the number of wells in the substrate, reaction times for each nucleotide and chemical blocking solutions, and wash cycle times. Typical cycle times for nucleotide reactions are between 30 seconds and 3 minutes per nucleotide at present. As enzymatic reactions become more efficient, e.g., almost instantaneous, the speed-limiting factor would be dependent on how long it takes to change reagents. As indicated in Table A, such write speeds can result in storage of 12.8 TB in less than 6 hours. In contrast, present HDD technologies can write about 1 TB of data in 1.5-2 hours, so the storage speed of the disclosed method is potentially about twice as fast as an HDD.

TABLE A

| | Packing | | | | | | | |
|---|---|---|---|---|---|---|---|---|
| Bases | Ratio | Well | | Substrate | Write | | Time to | |
| per | (Spacing:Well | Diameter | Speed | Size | Speed | GigaBytes | Write | TeraBytes |
| Cell | D) | (μm) | (sec) | (xy - μm) | (MBps) | (to write) | (hrs) | (equivalent) |
| 8 | 2:1 | 1 | 10 | 10000 | 0.078125 | 0.1 | 0.355556 | 0.0001 |
| 8 | 2:1 | 1 | 10 | 100000 | 7.8125 | 10 | 0.355556 | 0.01 |
| 8 | 2:1 | 0.1 | 10 | 100000 | 781.25 | 1000 | 0.355556 | 1 |
| 8 | 2.5:1 | 0.1 | 10 | 100000 | 625 | 800 | 0.355556 | 0.8 |
| 256 | 2.5:1 | 0.1 | 10 | 100000 | 625 | 25600 | 11.37778 | 25.6 |
| 256 | 2.5:1 | 0.1 | 5 | 100000 | 1250 | 25600 | 5.688889 | 25.6 |
| 256 | 5:1 | 1 | 10 | 100000 | 3.125 | 128 | 11.37778 | 0.128 |
| 256 | 5:1 | 1 | 5 | 100000 | 6.25 | 128 | 5.688889 | 0.128 |
| 256 | 5:1 | 0.1 | 10 | 100000 | 312.5 | 12800 | 11.37778 | 12.8 |
| 256 | 5:1 | 0.1 | 5 | 100000 | 625 | 12800 | 5.688889 | 12.8 |

In addition to the write speed, other significant advantages of the example method may include the following. First, throughput is parallel rather than linear or serialized. As noted, thousands of nanopore wells can be arranged in an array on a small silicon chip. Thus, separate data storage writes from disparate sources (e.g., multiple separate applications or multiple processors) could occur, reducing the need for threading or interleaving data writes to storage. Second, since the chips on which the DNA storage arrays are formed are very thin (e.g., 0.1-0.7 mm) and small in area, they can be stacked to provide dense storage capability in a small form factor.

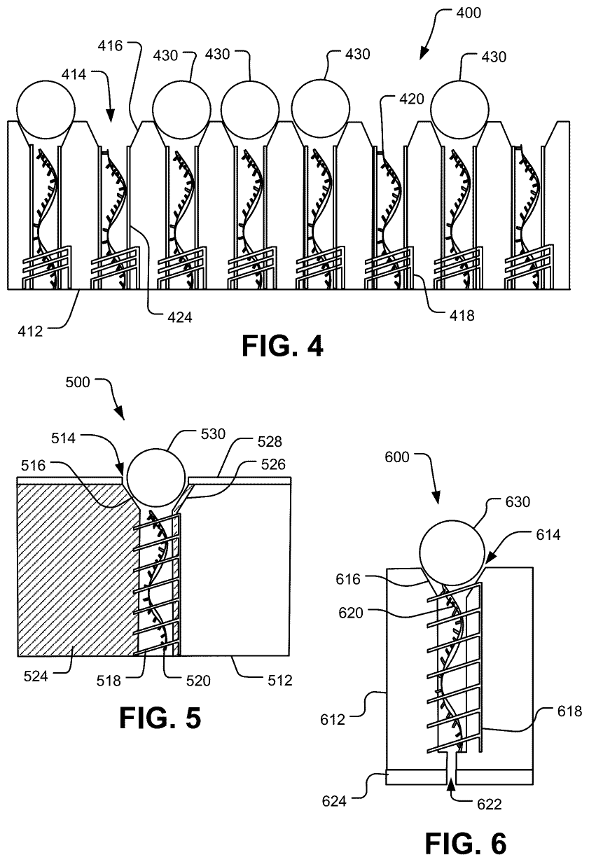

FIG. 4 depicts schematically an alternate example embodiment for implementation of nanopore wells 414 within a storage substrate 412 of a DNA synthesis apparatus 400. In this embodiment, as previously described with respect to FIG. 2, a single-stranded DNA 420 may be built up of successive nucleotides in each nanopore well 414. The nanopore wells 414 may be capped by MNPs 430, which rest on particle seats 416 formed about the top of each nanopore well 412, to prevent the entry of a nucleotide. Alternatively, the nanopore wells 414 may be uncapped to allow the entry of a nucleotide to bind with the free end of a single-stranded DNA 420. Each nanopore well 414 may also be at least partially surrounded by an electromagnetic coil 418 which, when energized, attracts a respective MNP 430 to engage the particle seat 416 of the nanopore well 414 and close it off. When electromagnetic coil 418 is deenergized, the corresponding MNP 430 may be removed (e.g., by washing it away or attracting it to a different surface), thereby opening the nanopore well 414 to accept another nucleotide.

Unlike in FIG. 2, in the DNA synthesis apparatus 400 of FIG. 4, the electromagnetic coil 418 does not extend the entire length of the nanopore well 414. In this embodiment, a sleeve 424, e.g., made of a ferromagnetic material, extends within and along the length of each nanopore well 414. The sleeve 418 may be a separate structure inserted into each nanopore well 414 or it may be a coating lining the surface of the interior of each nanopore well 414. The magnetic field generated by each of the electromagnetic coils 418 when energized may be imparted to the sleeve 418 to attract a respective MNP 430 to seat on the particle seat 416 of the nanopore well 414. The sleeves 418 may further focus the magnetic field at the openings of the nanotube wells 414 to better attract the MNPs 430. The electromagnetic coils 418 need not be located at the bases of the nanopore wells 414, but rather could be located at various other positions along the length of the nanopore wells 414, e.g., to facilitate ease of manufacture of the storage substrate 412. To maintain connection between the MNPs 430 and the nanopore wells 414, the electromagnetic coils 418 may need to be energized for an entire cycle of one or more nucleotide washes to prevent the MNPs 430 from unintentionally dislodging and allowing an improper nucleotide to bond to the single-stranded DNA 420 within the nanotube well 414.

FIG. 5 depicts schematically another alternate example embodiment for implementation of nanopore wells 514 within a storage substrate 512 of a DNA synthesis apparatus 500. In this embodiment, as previously described with respect to FIG. 2, a single-stranded DNA 520 may be built up of successive nucleotides in each nanopore well 514. The nanopore wells 514 may be capped by MNPs 530, which rest on particle seats 516 formed about the top of each nanopore well 512, to prevent the entry of a nucleotide. Alternatively, the nanopore wells 514 may be uncapped to allow the entry of a nucleotide to bind with the free end of a single-stranded DNA 520. Each nanopore well 514 may also be at least partially surrounded by an electromagnetic coil 518 which, when energized, attracts a respective MNP 530 to engage the particle seat 516 of the nanopore well 414 and close it off. When electromagnetic coil 518 is deenergized, the corresponding MNP 530 may be removed (e.g., by washing it away or attracting it to a different surface), thereby opening the nanopore well 514 to accept another nucleotide.

Similar to FIG. 2, in the DNA synthesis apparatus 500 of FIG. 5, the electromagnetic coil 418 extend the entire length of the nanopore well 514. However, in one form of this embodiment, the storage substrate 512 may be made of a high van der Wahls force material 524 or, in another form, the walls of the nanopore wells 514 within the storage substrate 512 may be covered with a high van der Waals force coating 528. Materials with high van der Waals forces may include, for example, graphene, graphite, or transition-metal dichalcogenides, For the purposes of this specification, “high van der Waals force” is considered a force equal to or greater than about 3 kJ/mole. Both of these alternatives are depicted in FIG. 5 for ease of presentation; however, it is more likely that only one or the other of these options would be implemented in a single configuration. In addition, the surface of the storage substrate 512 may be covered with a low van der Waals force material 528 or coating. Materials with low van der Waals forces may include, for example, polytetrafluoroethylene (PTFE), For the purposes of this specification, “low van der Waals force” is considered a force equal to or less than about 1 kJ/mole.

As before, the magnetic field generated by each of the electromagnetic coils 518 when energized may be used to attract a respective MNP 530 to seat on the particle seat 516 of the nanopore well 514. However, by using high van der Waals force materials 524 or high van der Waals force coatings 526 in the embodiment of FIG. 5, the MNPs 530 may remain seated in place due to the atomic forces without need to maintain a constant electric current through the electromagnetic coils 518 during an entire nucleotide wash cycle. This embodiment thus reduces the energy requirement for the DNA synthesis apparatus 500. Additionally, if the surface of the storage substrate 512 is coated with a low van der Waals force material 528, additional MNPs 530 are unlikely to collect on the surface of the storage substrate 512 and accidentally cover a nanopore well 514 that should not be covered in the present nucleotide solution cycle. In addition, selective electromagnetic coils 518 may be energized in an opposite electrical flow direction to create a repulsive magnetic force to overcome the attractive van der Waals forces and dislodge MNPs 530 from a capped position for a cycle in which those nanopore wells 514 are indicated for receipt of a nucleotide of the next nucleobase.

FIG. 6 depicts schematically a further alternate example embodiment for implementation of nanopore wells 614 within a storage substrate 612 of a DNA synthesis apparatus 600. In this embodiment, as previously described with respect to FIG. 2, a single-stranded DNA 620 may be built up of successive nucleotides in each nanopore well 614. The nanopore wells 614 may be capped by MNPs 630, which rest on particle seats 616 formed about the top of each nanopore well 612, to prevent the entry of a nucleotide. Alternatively, the nanopore wells 614 may be uncapped to allow the entry of a nucleotide to bind with the free end of a single-stranded DNA 620. Each nanopore well 614 may also be at least partially surrounded by an electromagnetic coil 618 which, when energized, attracts a respective MNP 630 to engage the particle seat 616 of the nanopore well 614 and close it off. When electromagnetic coil 618 is deenergized, the corresponding MNP 630 may be removed (e.g., by washing it away or attracting it to a different surface), thereby opening the nanopore well 614 to accept another nucleotide.

In the embodiment of FIG. 6, the nanopore well is further configured with an outlet read port 622, which is shown schematically as an opening in the base of the nanopore well 614 leading to a passage through a DNA read sensor 624. During synthesis of the single-stranded DNA 620, a chemical tether may be placed in each of the nanopore wells 614 as described above to provide an initial base for the DNA strand 620. The chemical tether may further hold the single-stranded DNA 620 within the nanopore well 614 and prevent it from migrating through the outlet read port 622. When there is a need to read the data encoded in the completed single-stranded DNA 620, the chemical tether can be chemically cleaved, allowing the single-stranded DNA to be removed from the nanopore well 614 through the outlet read port 622. Additionally, if the single-stranded DNA 620 was previously converted to a double-stranded DNA for storage, it may further need to be chemically separated to revert to single-stranded DNA 620 for reading. In other embodiments, the outlet read port 622 may be covered and then opened when a read request for the single-stranded DNA 620 in the nanopore well 614 is received. An example cover could be a micro-electronic mechanical system (MEMS) valve or switch, electronically addressable to open and close to provide or prevent access to the outlet read port 622. The DNA read sensor 624 at the outlet read port 622 may be configured to read the data from the DNA strands 620 using any of a variety of methods, for example, as described in M. Mansuripur, et al., “Information Storage and Retrieval using Macromolecules as Storage Media,” Optical Data Storage, OSA Technical Digest Series (Optica Publishing Group, 2003), paper TuC2.

It should be understood that any of the various features depicted in and described with respect to FIGS. 4, 5, and 6 can be provided in combination with each other. For example, the storage substrate in any example embodiment may be made of a high van der Waals force material; the surface of the storage substrate in any example embodiment may be coated with a low van der Waals force material; the electromagnetic coils in any example embodiment may extend the entire length of the nanopore wells, or may extend only a portion of the length of the nanopore wells along any location; the nanopore wells in any example embodiment may be lined with a ferromagnetic sleeve; and the nanopore wells in any example embodiment may include an outlet read port or be sized to store additional copies of the single-sided DNA strand (or corresponding double-sided DNA strands).

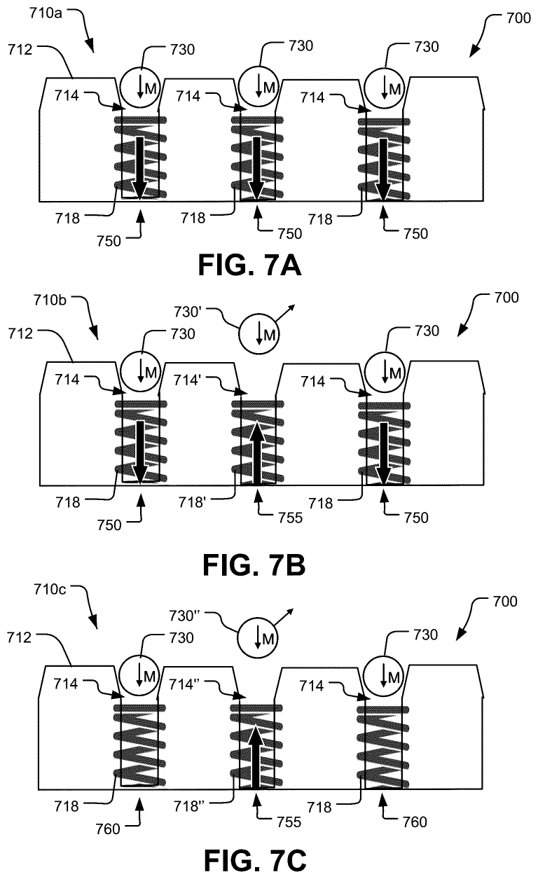

FIGS. 7A, 7B, and 7C schematically depict operations for energizing and deenergizing electromagnetic coils 718 in a storage substrate 712 of a DNA synthesis apparatus 700 to attract and release MNPs 730 to cover and uncover the nanopore wells 714. In a first energized configuration 710a shown in FIG. 7A, each of the electromagnetic coils 718 is energized in a current flow direction to create an attractive magnetic force 750 to draw the MNPs 730 to rest on the particle seats 716 at the top of the nanopore wells 714. Thus, in the first energized configuration 710a, all of the nanopore wells 714 in the storage substrate 712 are covered by a respective MNP 730. This configuration with all of the nanopore wells 714 covered may be the initial or starting configuration for the DNA synthesis apparatus 700 before each nucleotide binding cycle (or after four nucleotide binding cycles with four different nucleobases) as described in the process of FIG. 3.

In a second energized configuration 710b shown in FIG. 7B, two of the electromagnetic coils 718 are energized in a first current flow direction to create an attractive magnetic force 750 to draw the MNPs 730 to rest on the particle seats 716 at the top of the nanopore wells 714. However, a third electromagnetic coil 718′ is energized in a second current flow direction to create a repulsive magnetic force 755 to push away the MNP 730′ and thereby uncover a nanopore well 714′ that is designated for receipt of the next nucleotide solution flow in the data storage cycle. Thus, in the second energized configuration 710b, some of the nanopore wells 714 in the storage substrate 712 are covered by a respective MNP 730 and others of the nanopore wells 714′ are uncovered to accept a nucleotide.

A third energized configuration 710c is shown in FIG. 7C as an alternative energized configuration to FIG. 7B. The third energized configuration 710c may be desirable when the storage substrate 712 is made of or coated with a high van der Wahls force material as described previously with respect to FIG. 5. In the third energized configuration 710c, two of the electromagnetic coils 718 are in a deenergized state 760 without current flow in any direction. The MNPs 730 seated on the particle seats 716 of the nanopore wells 714 in the deenergized state 760 remain in place due to small forces such as friction, gravity, or van der Wahls forces as previously described. However, a third electromagnetic coil 718″ is energized in a second current flow direction to create a repulsive magnetic force 755 to push away the MNP 730″ and thereby uncover a nanopore well 714″ that is designated for receipt of the next nucleotide solution flow in the data storage cycle. Thus, in the third energized configuration 710c, some of the nanopore wells 714 in the storage substrate 712 are covered by a respective MNP 730 and others of the nanopore wells 714″ are uncovered to accept a nucleotide.

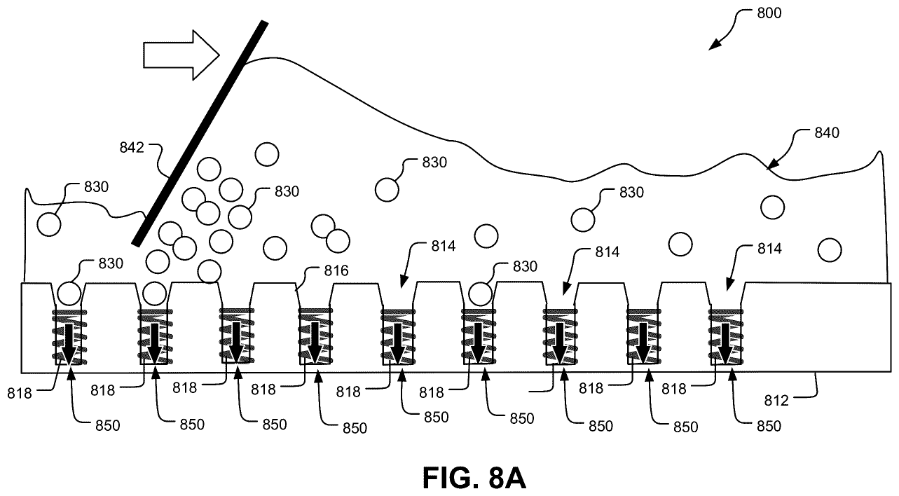

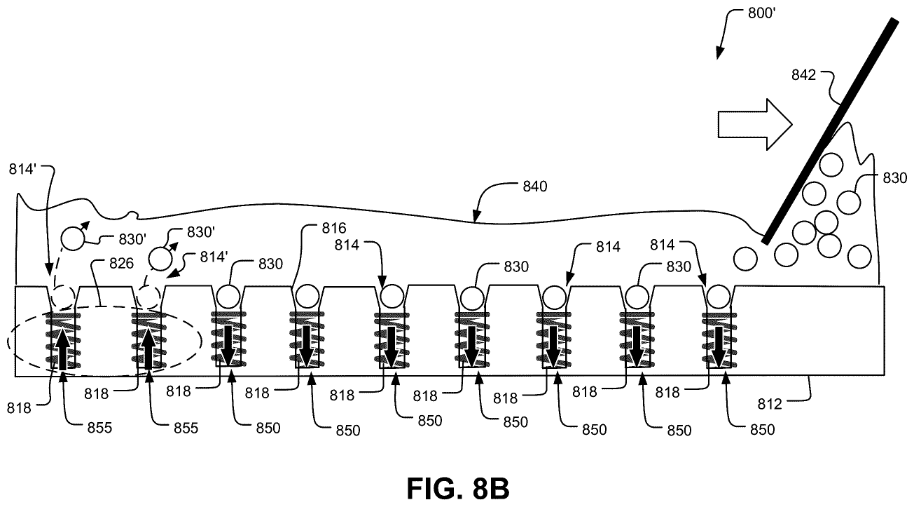

FIGS. 8A and 8B schematically depict one example method for covering all nanopore wells 814 in a storage substrate 812 of a DNA synthesis apparatus 800 with MNPs 830, for example, at the beginning of a nucleotide binding cycle (or after four nucleotide binding cycles with four different nucleobases) as described in the process of FIG. 3. In FIG. 8A, a fluid 840 containing or carrying MNPs 830 may be flowed over the storage substrate 812. In this example implementation, a wiper 842 may be used to sweep or push the fluid 840 across the surface of the storage substrate 812. However, many other microfluidic dispensing and flow control methods may alternatively be employed. As the wiper 842 sweeps the fluid 840, MNPs 830 within the fluid 840 may become more highly concentrated adjacent to the leading edge of the wiper 842. By increasing the concentration of the MNPs 830 near the wiper 842, it is more likely that the MNPs 830 will fill each of the nanopore wells 814 in the storage substrate 812. In addition, the electromagnetic coils 818 may be energized to create an attractive magnetic force 850 in each of the nanopore wells 814 to attract a corresponding MNP 830 in the fluid 840 and further ensure that MNPs 830 lodge in each particle seat 816 as the wiper 842 moves across the surface of the storage substrate 812.

FIG. 8B depicts the sweeping process of the wiper 842 at a later point in time. As discussed with respect to the method 300 of FIG. 3, in some implementations, it may be desirable to cover all of the nanopore wells 814 at the beginning of a nucleotide binding cycle and then remove MNPs 830 from certain nanopore wells 814′ that are designated to receive the next nucleotide solution with a desired nucleobase. (As previously discussed, in other implementations, it may be desirable to initially cover only those nanopore wells not scheduled to receive the nucleotide and leave the receiving nanopore wells open.) In order to speed the process of selective removal of MNPs 830, the electromagnetic coils 818′ of certain nanopore wells 814′ may be energized in an opposite current flow direction to create a repulsive magnetic force 855 to push selected MNPs 530′ off of the particle seats 816 and into the fluid 840. In this manner, the selected nanopore wells 814′ may be opened quickly in succession, e.g., one row or column at a time, following the sweep of the wiper 842 as it passes the position of each selected nanopore well 814. An intermediate washing step may be performed to remove the released MNPs 830′ in any remaining fluid 840. Alternatively, the introduction of the first nucleotide solution could be used to displace or wash away any released MNPs 830 on the surface of the storage substrate 812.

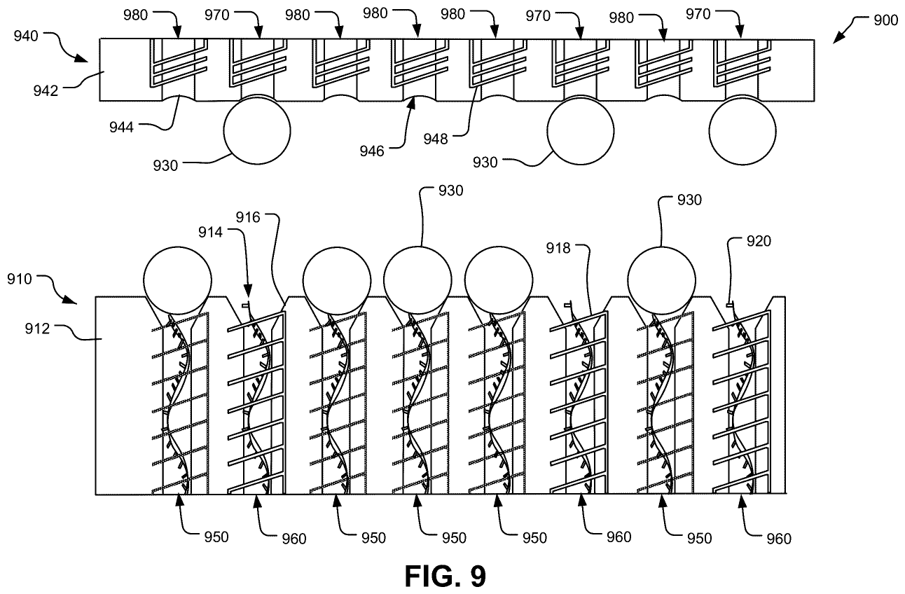

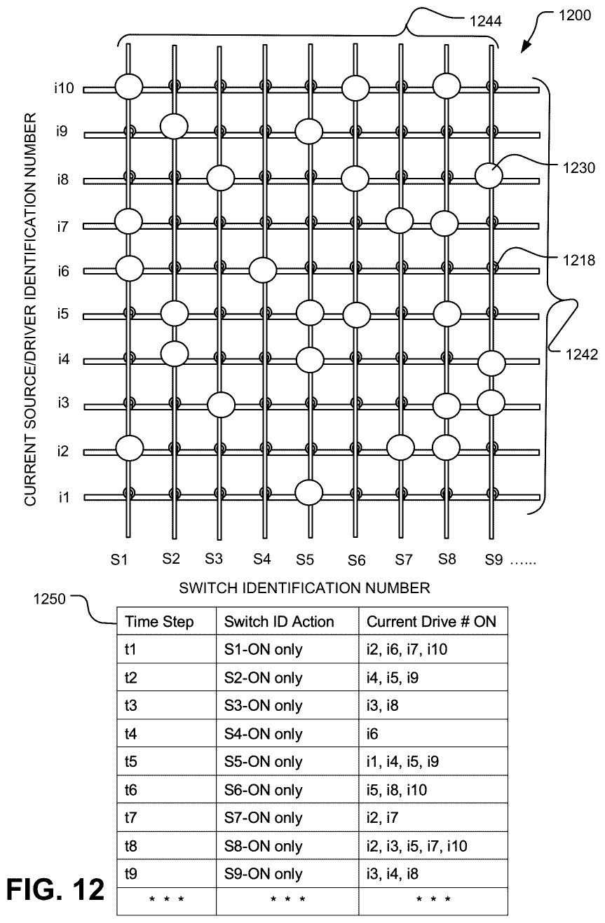

FIG. 9 is a schematic diagram of an example implementation of a DNA synthesis apparatus 900 used in example methods for synthesizing DNA strands, for example, the method described herein in below with respect to FIG. 12 for data storage. The DNA synthesis apparatus 900 may be composed of a lower plate 910 and an opposing upper plate 940. The lower plate 910 may include a storage substrate 912 populated by a plurality nanopore wells 914 arranged in an array pattern in which individual, single-stranded DNA 920 are formed and stored. The storage substrate 912 is depicted in FIG. 9 in cross section, thus showing a subset of only one row of parallel nanopore wells 914. However, in actuality, the storage substrate 912 would be composed of thousands of parallel rows of nanopore wells 914 with thousands of nanopore wells 914 in each row. A top edge of each nanopore well 914 may be designed as a particle seat 916 to receive a respective MNP 930 to cover the nanopore well 914. For example, the top edge of each nanopore well 914 may be beveled to form the particle seat 916 of receiving an MNP 930.

The storage substrate 912 may be made in a similar manner to computer chip manufacture as further described herein. In general, a silicon wafer base may support several interconnect layers, e.g., of silica or glass built on the silicon wafer, within which the nanopore wells 914 may be formed. In other implementations, the nanopore wells 214 may be formed in a layer of aluminum on top of a silicon wafer. The nanopore wells 214 may be on the order of 6 nm to 0.1 μm in diameter and about 25 nm deep. The nanopore wells 214 may be cylindrical or tapered frustoconical forms with down to an 80-degree slope from the top to the bottom beneath the particle seat 216.

Lower electromagnetic coils 918 may be formed around all or a portion of each respective nanopore well 914 and used to attract, release, or repel a respective MNP 930 as further described herein. The lower electromagnetic coils 918 may be formed by conductive traces deposited on each interconnect layer and electrically connected by conductive vias passing through each layer. For example, multiple interconnect layers may be built on the wafer and resist patterns may be used to define and maintain well openings, conductive traces paths, and vias. In another example, holes on the order of tenths of a micrometer may be bored into the interconnect layers built on the silicon wafer to form the nanopore wells 814. The bored holes may be bounded by the conductive trace forming the lower electromagnetic coils 918 around the nanopore wells 914. The silicon wafer may then be diced into chips in a similar manner to processor or memory chips. In some implementations, a single chip can be configured with millions of nanopore wells 214 to achieve multiple terabytes of capacity.

The MNPs 930 may be addressable controlled to cover and uncover their respective nanopore wells 914 to allow selective addition of a nucleotide to bind with a backbone chain of the single stranded DNA 920 in open nanopore wells 914. Nanopore wells 914 covered by respective MNPs 930 would not receive the selected nucleotide. Each of the four nucleotide types may be introduced to the storage substrate 912 in succession and cyclically to build the single-stranded DNA 920. In some implementations, the order of nucleotide deposition and bonding in each nanopore well 914 may be directed and controlled to encode digital data according to any of a number of mapping schemes that translate between ordered adjacent pairs of nucleotides in a single-stranded DNA 920 and digital data forms (e.g., binary, decimal, hexadecimal, etc.).

In the example implementation of the DNA synthesis apparatus 900 depicted in FIG. 9, an upper plate 940 is provided that opposes the lower plate 910 defining a small gap, e.g., 200 nm<5μ, between the lower plate 910 and the upper plate 940. The upper plate 940 may be formed of an attractor substrate 942, e.g., a silicon wafer supporting several interconnect layers of silica or glass, similar in construction to the storage substrate 912. The attractor substrate 942 includes a plurality of attractor posts 944 embedded therein. In some implementations the attractor posts 944 are formed of a ferromagnetic material. Exposed ends of each attractor post 944 may be concave in form to define a particle recess 946 for engaging a MNP 930. Each attractor post 944 corresponds to an opposing nanopore well 914 in the storage substrate 912. Thus, the attractor posts 944 are arranged in an array in the same pattern of the nanopore wells 912, with thousands of rows and columns resulting in potentially millions of attractor posts 944. The upper plate 940 is positioned in fine alignment with the lower plate 910 such that each attractor post 944 is registered in axial alignment with a respective, corresponding nanopore well 914 in the storage substrate 912.

Upper electromagnetic coils 948 may be formed around all or a portion of each respective attractor post 944 and used to attract, release, or repel a respective MNP 930 as further described herein. Additionally, the upper electromagnetic coils 948 may be formed by conductive traces deposited on each interconnect layer and electrically connected by conductive vias passing through each layer. For example, multiple interconnect layers may be built on the wafer and resist patterns may be used to define and maintain areas for the attractor posts 944, conductive traces paths, and vias. In another example, holes on the order of tenths of a micrometer may be bored into the interconnect layers built on the silicon wafer and filled with a ferromagnetic material, e.g., by a deposition process, to form the attractor posts 944. The attractor posts 944 may be bounded by the conductive traces forming the upper electromagnetic coils 948.

When an upper electromagnetic coil 948 is energized with current traveling in a first direction, an attractive magnetic force 970 is created and a free MNP 930 located within a short range will be attracted to the attractor post 944 and seat within the corresponding particle recess 946. If the upper electromagnetic coil 948 is not energized, the attractor post 944 has a neutral magnetic field 980 and thus does not attract an MNP 930. When the upper plate 940 is provided in conjunction with the lower plate 910, the lower electromagnetic coils 918 and the upper electromagnetic coils 948 may be energized and deenergized in cooperation to open and close desired nanopore wells 914 by seating and unseating corresponding MNPs 930.

As shown in FIG. 9, when selected lower electromagnetic coils 918 in the storage substrate are energized to create an attractive magnetic force 950, an MNP 930 is drawn into the particle seat 916 of the coaxial nanopore well 914 and held in place to seal the nanopore well 914. The opposing upper magnetic coil 948 is not energized and has a neutral magnetic field 980. However, for nanopore wells 914 that are designated to receive a nucleotide in the next cycle, the corresponding lower electromagnetic coils 918 are deenergized, resulting in a neutral magnetic field 960. Simultaneously, the opposing upper magnetic coils 948 are energized to create an attractive magnetic field 970 and pull the MNP 930 away from the nanopore well 914 to seat in the particle recess 946 of an opposing attractor post 942. Thus, by energizing and deenergizing opposing lower electromagnetic coils 918 and upper electromagnetic coils 948, the nanopore wells 914 can be addressable opened and closed to accept and block nucleotides to write or encode unique DNA strands 920.

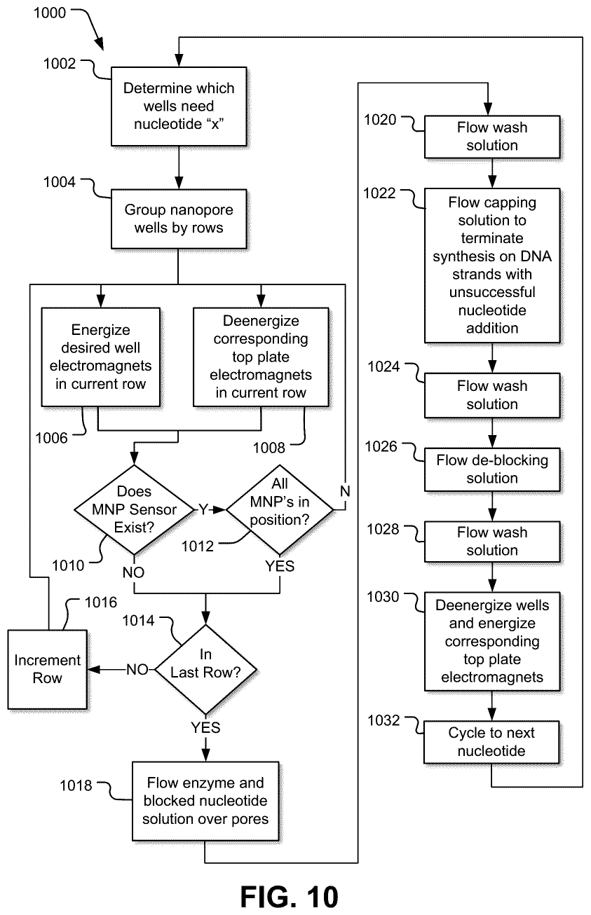

An example process 1000 for using the DNA synthesis apparatus 900 of FIG. 9 is presented in the flow diagram of FIG. 10. Initially, in a determining operation 1002, a control system, for example, including a computing processor, memory, and software instructions, may determine which nanopore wells in the DNA synthesis apparatus should receive nucleotide “X”, i.e., one of the four nucleotides to be introduced cyclically. Next, in a grouping operation 1004, the nanopore wells in the lower plate and corresponding attractor posts in the upper plate are grouped in opposing, aligned rows for sequential assignment of MNP exchange by the control system. Two operations are then performed in tandem. In an energizing operation 1006 the lower electromagnetic coils corresponding to nanopore wells in the bottom plate that should be capped by a MNP are energized. Correspondingly, in a deenergizing operation 1008, the upper electromagnetic coils around the attractor posts in the upper plate above corresponding nanopore wells that need to be covered are deenergized so the MNPs will be attracted by the electromagnetic fields generated around the opposing nanopore wells and travel to the nanopore wells to seat thereon and cover them.

In one example, optional implementation, a query operation 1010 may be performed to determine whether the control system is configured to determine whether all of the nanopore wells that need to be capped are actually covered by an MNP. This determination may be made, for example, by monitoring for a change in current flow, e.g., greater resistance, due to the presence of an MNP interacting with the lower electromagnetic coil when seated. If the control system can make such a determination, a further query operation 1012 may be performed to determine whether each of the nanopore wells in the addressed row was actually covered by an MNP. If any nanopore wells that were assigned to be covered were not, the process returns to tandem energizing operation 1006 and deenergizing operation 1008 to again attempt to cover the assigned nanopore wells in the row. If it Is determined in query operation 1010 that the control system does not have MNP seating sensing ability or in query operation 1012 that all assigned nanopore wells in the addressed row have been covered by a respective MNP, the process moves to query operation 1014 to determine whether the sequential assignment of MNPs to cover nanopore wells has reached the last grouped row in the array. If not, the control system increments to the next row and the process returns to the tandem energizing operation 1006 and deenergizing operation 1008 to appropriately seat MNPs on nanopore wells assign for closure by the control system.

If, instead, the last row in the array has been reached and assigned nanopore wells are covered, the process 1000 moves to a first flowing operation 1018 in which a binding enzyme and a blocked nucleotide solution of nucleotide “X” is flowed over the nanopore wells in the storage substrate of the lower plate. The binding enzyme assists in the binding of nucleotide “X” to the free ends of DNA strands in open nanopore wells. A second end of each nucleotide is “blocked” by a chemical compound to prevent the nucleotides from binding with each other and creating long, homopolymer chains rather than adding a single nucleotide to the exposed DNA strands. Next, in a second flowing operation 1020, a wash solution is flowed over the lower plate to wash away any excess, unbound nucleotides and binding enzyme out of any open nanopore wells and from the surface of the storage substrate. In a third flowing operation 1222, a capping solution is flowed over the lower plate. The capping solution includes a chemical cap structured to terminate synthesis on any DNA strands in nanopore wells that failed to successfully bind with nucleotide “X”. These DNA strands will not be able to bind to any further nucleotides and will be considered data write errors when subsequently read.

A fourth flowing operation 1024 next flows another wash solution over the lower plate to wash away any excess capping solution out of any open nanopore wells and off the surface of the storage substrate. A fifth flowing operation 1026 then flows a de-blocking solution to remove the blocking chemical compound from the DNA strands of the open nanopore wells. This will allow the DNA strands to bind with the next nucleotide should the nanopore well remain open during the next cycle. Note that the deblocking solution will not have any effect on the DNA strands that have been chemically capped; these DNA strands are inert and will not accept any further nucleotides in any future cycle. A sixth flowing operation 1028 then flows a further wash solution over the lower plate to wash any excess deblocking solution out of any open nanopore wells and off the surface of the storage substrate.

After the storage substrate is washed clean, the process 1000 deenergizes all of the lower electromagnetic coils and energizes all of the upper electromagnetic coils to attract the MNPs to the attractor posts in the upper plate and uncover all of the nanopore wells. Alternatively, the control system may addressable deenergize only the lower electromagnetic coils corresponding to the covered nanopore wells and energize only the upper magnetic coils around attractor posts above the capped nanopore wells to attract the MNPs to the upper plate and uncover the covered nanopore wells. Finally, in cycling operation 1032, the process cycles forward to close appropriate nanopore wells with MNPs to restrict introduction of the next nucleotide for binding with only selected DNA strands according to the DNA synthesis scheme in determination operation 1002.

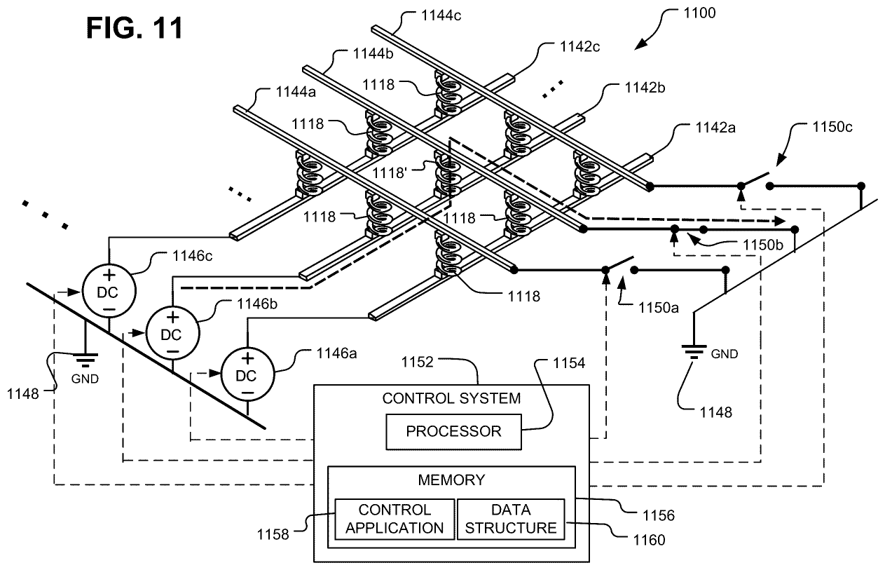

An example schematic diagram of a driving and switching circuit 1100 for addressable energizing and deenergizing electromagnetic coils in a storage substrate or in each of a lower plate and an upper plate, e.g., in the DNA synthesis apparatus 900 of FIG. 9, is depicted in FIG. 11. The circuit 1100 includes a plurality of current traces 1142a/b/c and a plurality of switch traces 1144a/b/c arranged perpendicular to each other to form a grid. In the example of FIG. 11, only three current traces 1142a/b/c and three switch traces 1144a/b/c are shown for simplicity and clarity; however, there would be thousands of each in the circuit 1100 for controlling a DNA synthesis apparatus. The current traces 1142a/b/c and the switch traces 1144a/b/c are separated from each other by a plurality of electromagnetic coils 1118 that provide electrical connections between the current traces 1142a/b/c and the switch traces 1144a/b/c at each geometric intersection of the current traces 1142a/b/c and the switch traces 1144a/b/c (e.g., gid intersections when the circuit 1100 is considered in a plan view).

One end of each of the current traces 1142a/b/c is electrically connected to a respective voltage source 1146a/b/c, which may in turn be connected to ground 1148. Similarly, one end of each of the switch traces 1144a/b/c is connected to first contact of a respective switch 1150a/b/c. Each voltage source 1146a/b/c and switch 1150a/b/c is connected to a controller or control system 1152 that generates signals to individually energize respective voltage sources 1146a/b/c and open and close each switch 1150a/b/c. A second contact of each switch is further connected to ground 1148 to complete a closed circuit when the respective switch 1150a/b/c is closed by the control system 1152. The control system 1152 may include a computing processor 1154 and a memory 1156. The memory 1156 may further include a control application 1158 stored thereon for execution by the processor 1154 to perform the switching operations. The memory 1156 may also include a data structure 1160 stored thereon for access by the control application 1158 to identify the nanopore wells to be opened and closed for nucleotide binding and thereby inform the switching operations. In another example embodiment, a single voltage source could be used and the control system 1152 could alternately control additional respective switches between the voltage source and each of the current traces 1142a/b/c to direct current to specific current traces 1142a/b/c.

Current only flows through a current trace 1142a/b/c, and correspondingly through an electromagnetic coil 1118 to create a magnetic field, when a voltage source 1146a/b/c is activated and a corresponding switch 1150a/b/c connected to a corresponding switch trace 1144a/b/c is closed by the control system 1152. The control system 1152 may be a computing device with a processor 1154 and memory 1156 including a software control application 1158 for managing the voltage sources 1146a/b/c and switches 1150a/b/c to build the DNA strands with desired orders of nucleotides, e.g., for data storage, according to sequence instruction data stored in a data structure 1160 in the memory 1156. The data structure 1160 is organized to describe data to be stored in the array of nanopore wells in the form of a DNA sequence, or an intrinsic DNA sequence, and the control application 1158 interprets the data structure to control the current flow and switching to open and close the nanopore wells for “writing” the nucleotides. As depicted in the example of FIG. 11, switch 1152b has been closed by the controller 1152, allowing current to flow from the voltage source, through the current trace 1142b, through the corresponding electromagnetic coil 1118′, and through the corresponding switch trace 1144b to ground 1148. In this example, the electromagnetic coil 1118′ bounds a corresponding nanopore well (not shown in FIG. 11) and creates a magnetic field that may either attract or repel (depending upon the direction of the current) an MNP to close or open the nanopore well.

FIG. 12 schematically depicts a switch map 1200 for an example of controlled switching to addressable energize electromagnetic coils 1218 in an array to attract, release, or repel MNPs 1230 to cover or uncover nanopore wells for blocking or receipt of nucleotides. The switch map 1200 presents a plurality of current traces 1242 and a plurality of switch traces 1244 arranged perpendicular to each other in to form a grid. A plurality of electromagnetic coils electrically connect the current traces 1242 and the switch traces 1244 at each grid intersection. Beneath the switch map 1200, an example switch table 1250 is presented as an aid in understanding an example switch control scheme performed by a controller (e.g., the controller 1052 in FIG. 10).

As indicated in the switch table 1250, at time t1 the controller closes the switch connected to switch trace S1 and energizes a voltage source to drive current along current traces i1, i6, i7, and i10, four electromagnetic coils 1218 along switch trace S1 will be energized to attract respective MNPs 1230 to cover the corresponding nanopore wells as shown in the switch map 1200. After an effective time, e.g., an adequate period for attraction and seating of a corresponding MNPS 1230 by energized electromagnetic coils 1218, the current i1 may be turned off and the switch of switch trace S1 may be closed. The MNPs 1230 remain seated in place, e.g., by magnetic attraction to ferromagnetic materials lining the nanopore wells or by van der Wahls forces as previously described herein. Next, as indicated in the switch table 1250, at time t2 the controller closes the switch connected to switch trace S2 and energizes a voltage source to drive current along current traces i4, i5, and i9, three electromagnetic coils 1218 along switch trace S2 will be energized to attract respective MNPs 1230 to cover the corresponding nanopore wells as shown in the switch map 1200. Again, after an effective time, the current i2 may be turned off, the switch of switch trace S2 may be closed, and the MNPs 1230 along switch trace S2 remain seated in place.

As shown in FIG. 12, this same process occurs successively (for times t1-19, etc.) across the entire array for each combination of current traces (i1-i10, etc.) and switch traces (S1-S9, etc.) to assignably seat MNPS 1230 over nanopore wells that are identified in the write instructions for building the DNA strands (e.g., for data storage) as not to receive the nucleotide in the next nucleotide flow across the DNA synthesis apparatus. As described above, seated MNPs 1230 may be removed and replaced between each nucleotide flow or subsets of seated MNPs 1230 may be removed to open new nanopore wells through each of four cycles of nucleotide flows—all nanopore wells would be open for the last nucleotide flow—but, as described above, only the most recently opened nanopore wells would have DNA strands without chemical blockers to bind the successive nucleotides. Successive opening of subsets of the nanopore wells can be achieved, for example using the DNA synthesis apparatus 900 of FIG. 9 with the upper plate 940.

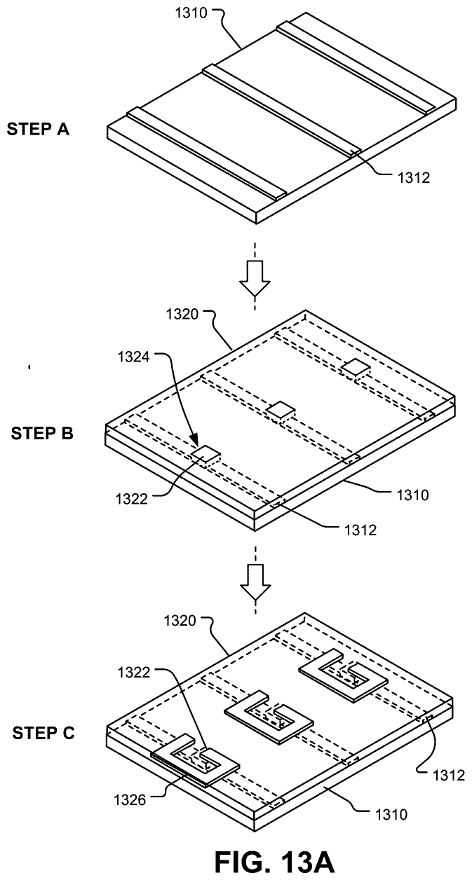

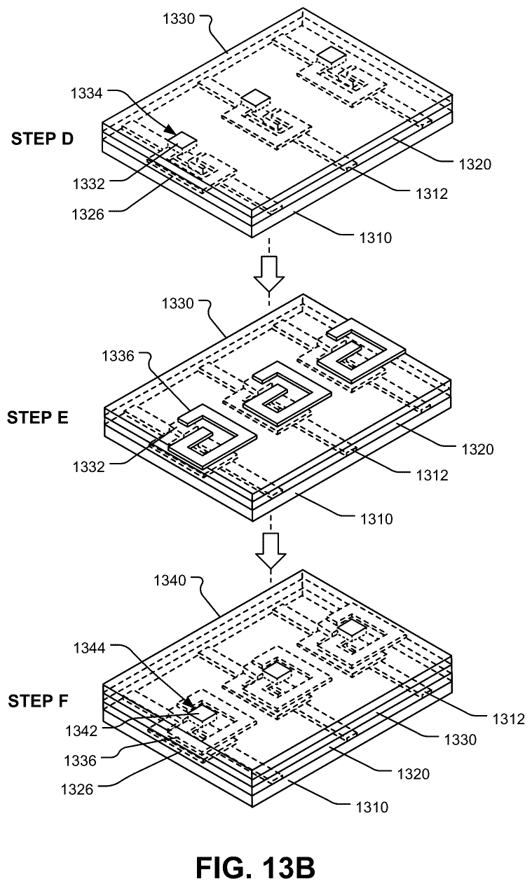

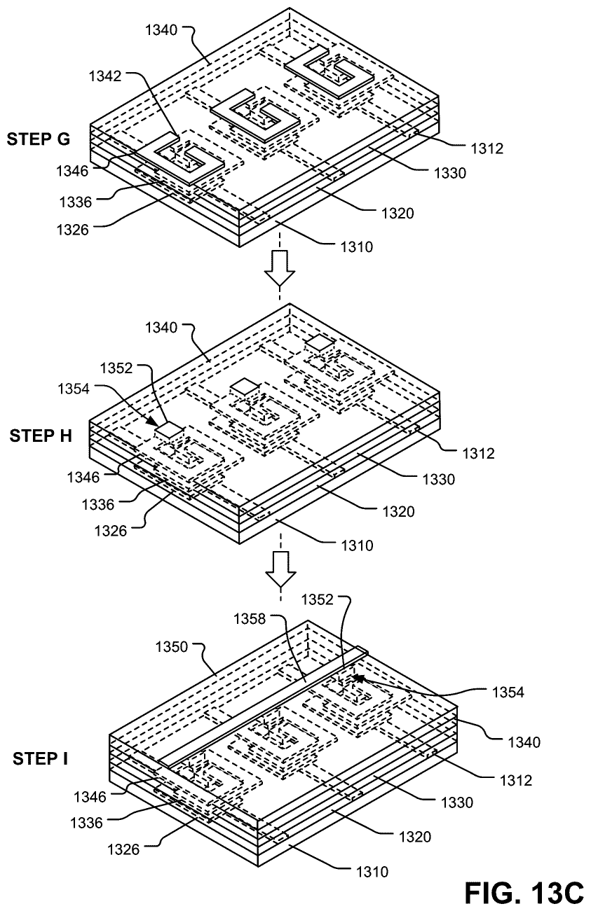

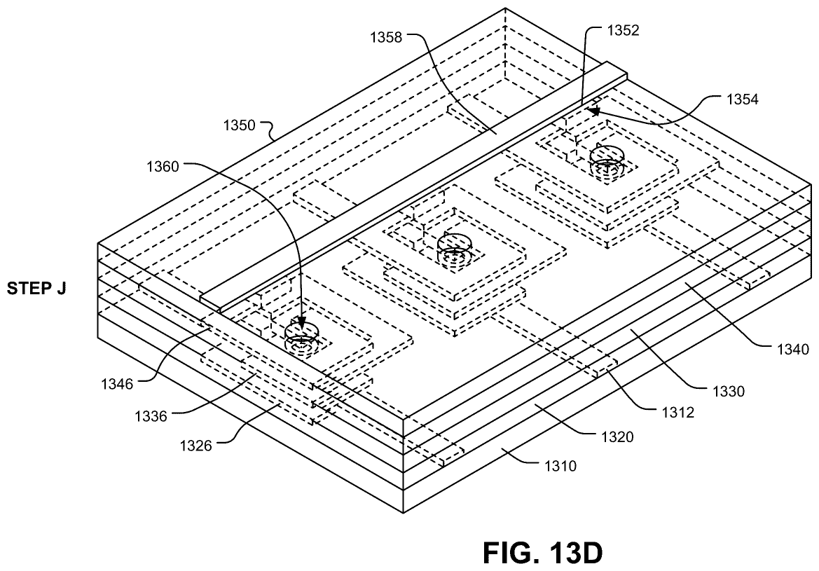

FIGS. 13A-13D illustrate as a storyboard the structure of an example upper or lower plate of a DNA synthesis apparatus as described herein. As depicted in the schematic labeled Step A of FIG. 13A, a first substrate layer 1310, e.g., a silicon wafer or die, forms the base of the DNA synthesis apparatus. A plurality of conductive current traces 1312 are formed on the first substrate layer 1310, for example, by lithographic design, deposition, and etching or other typical methodologies for building circuits on a substrate. The current traces 1312 may be composed of copper, silver, gold, aluminum, or other highly conductive metal. As depicted in the schematic labeled Step B of FIG. 13A, a second substrate layer 1320 may be formed on the surface of the first substrate layer 1310 and covers the current traces 1312. The second substrate layer 1320 may be composed of a typical insulating material for integrated circuits, for example, a silicon or silicon-oxide dielectric, or a thin film polymer. Electrically conductive first connectors 1322 are formed in second layer vias 1324 extending through the second substrate layer 1320. The first connectors 1322 seat upon and electrically connect to respective current traces 1312 and are exposed through the top surface of the second substrate layer 1320, which defines the second layer vias 1324.

The second layer vias 1324 may be formed by typical integrated circuit manufacture processes, e.g., by placing a resist pattern over the first substrate layer 1310 and current traces 1312 to displace the second substrate layer 1320 from the areas of the second layer vias 1324 when the second substrate layer 1320 is coated over the first substrate layer 1310. The resist material is then removed, e.g., by a chemical etch process, and the first connectors 1322 are formed by depositing a conductive metal within the now-formed second layer vias 1324 to connect to the current traces 1312 and extend to the outer surface of the second substrate layer 1320.

Next, as depicted in the schematic labeled Step C in FIG. 13A, first coil windings 1326 may be formed on the surface of the second substrate layer 1320, with a first end of each first coil winding 1326 in physical and electrical contact with the top surface of a respective first connector 1322 exposed within a respective second layer via 1324. The first coil winding 1326, as with all the electrical structures in the upper and lower plates of the DNA synthesis apparatus, may be a composed of a conductive metal such as copper, silver, gold, or aluminum. The first coil windings 1326 may be formed by a metal plating process within channels designed within a temporary resist layer that is chemically etched away after the first coil windings 1326 are plated. The first coil windings 1326 form an open loop with a second end of each of the first coil windings 1326 separated by a gap from the first end connected to the first connectors 1322.

Next, as depicted in the schematic labeled Step D of FIG. 13B, a third substrate layer 1330 may be formed on the surface of the second substrate layer 1320 and also cover the first coil windings 1326. The third substrate layer 1330 may again be composed of a typical insulating material for integrated circuits, for example, a silicon or silicon-oxide dielectric, or a thin film polymer. Electrically conductive second connectors 1332 are formed in third layer vias 1334 extending through the third substrate layer 1330. The second connectors 1332 seat upon and electrically connect to respective second ends of the first coil windings 1326 and are exposed through the top surface of the third substrate layer 1330, which defines the third layer vias 1334.

The third layer vias 1334 may be formed, as before, by typical integrated circuit manufacture processes, e.g., by placing a resist pattern over the second substrate layer 1320 and first coil windings 1326 to displace the third substrate layer 1330 from the areas of the third layer vias 1334 when the third substrate layer 1330 is coated over the second substrate layer 1320. The resist material is then removed, e.g., by a chemical etch process, and the second connectors 1332 are formed by depositing a conductive metal within the now-formed third layer vias 1334 to connect to the first coil windings 1326 and extend to the outer surface of the third substrate layer 1330.

Next, as depicted in the schematic labeled Step E in FIG. 13B, second coil windings 1336 may be formed on the surface of the third substrate layer 1330, with a first end of each second coil winding 1336 in physical and electrical contact with the top surface of a respective second connector 1332 exposed within a respective third layer via 1334. The second coil winding 1336, may be a composed of a conductive metal such as copper, silver, gold, or aluminum. The second coil windings 1336 may be formed by a metal plating process within channels designed within a temporary resist layer that is chemically etched away after the second coil windings 1336 are plated. The second coil windings 1336 form an open loop with a second end of each of the second coil windings 1336 separated by a gap from the first end connected to the second connectors 1332. In some example implementations, the second ends of the second coil windings 1336 may be positioned directly above the locations of respective first connectors 1322. The second coil windings 1336 should be positioned in conjunction with the first coil windings 1326 such that the loops formed by each should together define and bound a central area in each of the substrate layers in which a respective nanopore well can later be formed.

As depicted in the schematic labeled Step F of FIG. 13B, a fourth substrate layer 1340 may be formed on the surface of the third substrate layer 1330 and also cover the second coil windings 1336. The fourth substrate layer 1340 may again be composed of a typical insulating material for integrated circuits, for example, a silicon or silicon-oxide dielectric, or a thin film polymer. Electrically conductive third connectors 1342 are formed in fourth layer vias 1344 extending through the fourth substrate layer 1340. The third connectors 1342 seat upon and electrically connect to respective second ends of the second coil windings 1336 and are exposed through the top surface of the fourth substrate layer 1340, which defines the fourth layer vias 1344.

The fourth layer vias 1344 may be formed, as before, by typical integrated circuit manufacture processes, e.g., by placing a resist pattern over the third substrate layer 1330 and second coil windings 1336 to displace the fourth substrate layer 1340 from the areas of the fourth layer vias 1344 when the fourth substrate layer 1340 is coated over the third substrate layer 1330. The resist material is then removed, e.g., by a chemical etch process, and the third connectors 1342 are formed by depositing a conductive metal within the now-formed fourth layer vias 1344 to connect to the second coil windings 1336 and extend to the outer surface of the fourth substrate layer 1340.

Further, as depicted in the schematic labeled Step G in FIG. 13C, third coil windings 1346 may be formed on the surface of the fourth substrate layer 1340, with a first end of each third coil winding 1346 in physical and electrical contact with the top surface of a respective third connector 1342 exposed within a respective fourth layer via 1344. The third coil winding 1346, may be a composed of a conductive metal such as copper, silver, gold, or aluminum. The third coil windings 1346 may be formed by a metal plating process within channels designed within a temporary resist layer that is chemically etched away after the third coil windings 1346 are plated. The third coil windings 1346 form an open loop with a second end of each of the third coil windings 1346 separated by a gap from the first end connected to the third connectors 1342. In some example implementations, the second ends of the third coil windings 1346 may be positioned directly above the locations of respective second connectors 1332. The third coil windings 1346 should be positioned in conjunction with the first coil windings 1326 and the second coil windings 1336 such that the loops formed by each should together define and bound a central area in each of the substrate layers in which a respective nanopore well can later be formed. In one example, the third coil windings 1346 may have the same shape and footprint as, and be positioned directly over, the first coil windings 1326.

As depicted in the schematic labeled Step H of FIG. 13C, a fifth substrate layer 1350 may be formed on the surface of the fourth substrate layer 1340 and also cover the third coil windings 1346. The fifth substrate layer 1350 may again be composed of a typical insulating material for integrated circuits, for example, a silicon or silicon-oxide dielectric, or a thin film polymer. Electrically conductive fourth connectors 1352 are formed in fifth layer vias 1354 extending through the fifth substrate layer 1350. The fourth connectors 1352 seat upon and electrically connect to respective second ends of the third coil windings 1346 and are exposed through the top surface of the fifth substrate layer 1350, which defines the fifth layer vias 1354.

The fifth layer vias 1354 may be formed, as before, by typical integrated circuit manufacture processes, e.g., by placing a resist pattern over the fourth substrate layer 1340 and third coil windings 1346 to displace the fifth substrate layer 1350 from the areas of the fifth layer vias 1354 when the fifth substrate layer 1350 is coated over the fourth substrate layer 1340. The resist material is then removed, e.g., by a chemical etch process, and the fourth connectors 1352 are formed by depositing a conductive metal within the now-formed fifth layer vias 1354 to connect to the third coil windings 1346 and extend to the outer surface of the fifth substrate layer 1350. It may be appreciated that additional dielectric substrate layers and coil windings may be built up, layer by layer, to provide sufficient windings in a coil to create the necessary magnetic fields to attract the MNPs. Further, the substrate layers may be built up to provide sufficient depth to accommodate a desired depth of the nanopore wells.

As depicted in the schematic labeled Step I of FIG. 13C, a conductive switch trace 1358 is formed on the fifth substrate layer 1350, for example, by lithographic design, deposition, and etching or other typical methodologies for building circuits on a substrate. The switch traces 1358 extends across the surface of the fifth substrate layer 1350 and is in physical and electrical contact with each of the fourth connectors 1352 exposed in the surface of the fifth substrate layer 1350. The switch trace 1358 may be composed of copper, silver, gold, aluminum, or other highly conductive metal. While only one switch trace 1358 is depicted in STEP I of FIG. 13C, in actuality, thousands of switch traces 1358 would be formed perpendicular to corresponding thousands of current traces 1312 to create a grid array for addressable control of electromagnetic coils surrounding each nanopore well.

FIG. 13D depicts a portion of a completed lower plate of a DNA synthesis apparatus with nanopore wells 1360 formed within the bounds of the first, second, and third coil windings 1326, 1336. 1346. In one example implementation, the nanopore wells 1360 may be drilled within the bounds of the first, second, and third coil windings 1326, 1336. 1346. In another example implementation, the nanopore wells 1360 may be formed by stack of resist layers in each substrate that are of a different resist material as used for marking the vias. By using a different resist material to define the nanopore wells 1260, the chemical etch processes for the vias and the coil windings may not impact the resist material defining the nanopore wells 1360. Once all of the substrate layers are in place and the switch traces 1358 are formed on the surface of the top substrate layer, a different etch chemical can be applied to remove the resist layers defining the nanopore wells 1360 and opening them to the top surface of the lower plate. In the case of the top plate, a similar process may be invoked to define nanopores that are then filled with a ferromagnetic material to form the attractor posts. Alternatively, the attractor posts could be formed using layer by layer deposition as each dielectric substrate layer is built on the top plate.

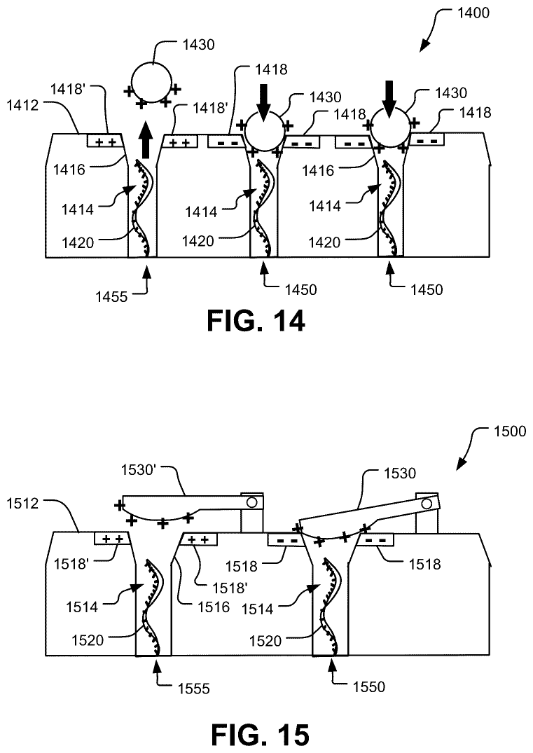

FIG. 14 depicts schematically a further example embodiment for implementation of nanopore wells 1414 within a storage substrate 1412 of a DNA synthesis apparatus 1400. In this embodiment, a single-stranded DNA 1420 may be built up of successive nucleotides in each nanopore well 1414. The nanopore wells 1414 may be capped by electrostatically charged particles 1430, e.g., polystyrene beads, that are attracted and repelled by electric fields in lieu of magnetic fields. The electrostatically charged particles 1430 rest on particle seats 1416 formed about the top of each nanopore well 1414, to prevent the entry of a nucleotide. Alternatively, the nanopore wells 1414 may be uncapped to allow the entry of a nucleotide to bind with the free end of a single-stranded DNA 1420. Each nanopore well 1414 may also be at least partially bounded by electric traces 1418 which, when energized with an opposing charge to the charge of the electrostatically charged particles 1430, attract a respective electrostatically charged particles 1430 to engage the particle seat 1416 of the nanopore well 1414 and close it off.

The electric traces 1418 may be formed in a grid pattern in a form and manner similar to the current traces and switch traces described with respect to FIGS. 11-13D above. The electric traces 1418 may be underneath the surface of the storage substrate 1412 to avoid direct contact with the electrostatically charged particles 1430, which could cause the electrostatically charged particles 1430 to discharge their charge. When electric traces 1418′ around particular nanopore wells 1414 are energized with a same charge as the electrostatically charged particles 1430, the electrostatically charged particles 1430 are repelled and do not seat on the corresponding nanopore well 1414, thereby maintaining the opening to the nanopore well 1414 to accept another nucleotide. Excess electrostatically charged particles 1430 not attracted to and not seated upon a nanopore well 1414 may be washed away in a wash cycle.

FIG. 15 depicts schematically yet another example embodiment for implementation of nanopore wells 1514 within a storage substrate 1512 of a DNA synthesis apparatus 1500. In this embodiment, a single-stranded DNA 1520 may be built up of successive nucleotides in each nanopore well 1514. The nanopore wells 1514 may be capped and uncapped by an electromechanical cap or valve. In the example of FIG. 15, a hinged valve 1530 is electrostatically charged and is attracted and repelled by electric fields in lieu of magnetic fields. The electrostatically charged hinged valves 1530 rest on valve seats 1516 formed about the top of each nanopore well 1514, to prevent the entry of a nucleotide. Alternatively, the nanopore wells 1514 may be uncapped to allow the entry of a nucleotide to bind with the free end of a single-stranded DNA 1520. Each nanopore well 1514 may also be at least partially bounded by electric traces 1518 which, when energized with an opposing charge to the charge of the electrostatically charged hinge valve 1530, attract an opposing hinged valve 1530 to engage the valve seat 1416 of the nanopore well 1514 and close it off.

The electric traces 1518 may be formed in a grid pattern in a form and manner similar to the current traces and switch traces described with respect to FIGS. 11-13D above. The electric traces 1518 may be underneath the surface of the storage substrate 1512 to avoid direct contact with the electrostatically charged hinged valve 1530, which could cause the electrostatically charged hinged valve 1430 to discharge its charge. When electric traces 1518′ around particular nanopore wells 1514 are energized with a same charge as the electrostatically charged hinged valve 1530, the electrostatically charged hinged valve 1530 is repelled and does not seat on the corresponding nanopore well 1514, thereby maintaining the opening to the nanopore well 1514 to accept another nucleotide.

All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the structures disclosed herein, and do not create limitations, particularly as to the position, orientation, or use of such structures. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. The example drawings are for purposes of illustration only and the dimensions, positions, order and relative sizes reflected in the drawings attached hereto may vary.

The above specification, examples and data provide a complete description of the structure and use of example embodiments of the invention as defined in the claims. Although various embodiments of the claimed invention have been described above with a certain degree of particularity, or with reference to one or more individual embodiments, other embodiments using different combinations of elements and structures disclosed herein are contemplated, as other iterations can be determined based upon the teachings of the present disclosure. It is intended that all matter contained in the above description and shown in the accompanying drawings shall be interpreted as illustrative only of particular embodiments and not limiting. Changes in detail or structure may be made without departing from the basic elements of the invention as defined in the following claims.

What is claimed is:

1. A method for serial and contemporaneous synthesis of disparate deoxy-ribonucleic acid (DNA) or ribonucleic acid (RNA) strands in an array of wells defined within a substrate, the method comprising
closing a first subset of wells of the array of wells defined within the substrate, wherein each well in the array of wells contains a precursor nucleotide chain, and the first subset of wells is designated as proscribed from receiving a nucleotide of a specified nucleobase type for a cycle of the method;
flowing a solution of a binding reaction enzyme and nucleotides of the specified nucleobase type bound with a corresponding chemical blocker over the array of wells;
receiving nucleotides of the specified nucleobase type in each of a second subset of wells of the array of wells that are open and designated to receive the nucleotide of the specified nucleobase type; and
binding received nucleotides of the specified nucleobase type with assistance of the binding reaction enzyme to corresponding precursor nucleotide chains in the second subset of wells.

2. The method of claim 1, wherein closing the first subset of wells further comprises energizing electromagnetic coils defined respectively around each well of the first subset of wells to attract corresponding magnetic nanopore particles (MNPs) to cover the first subset of wells.

3. The method of claim 1 further comprising opening the second subset of wells by deenergizing electromagnetic coils defined respectively around each well of the second subset of wells to release respective magnetic nanopore particles (MNPs) covering the second subset of wells.

4. The method of claim 2 further comprising, while closing the first subset of wells, opening the second subset of wells by deenergizing electromagnetic coils defined respectively around the each well of the second subset of wells to release respective MNPs covering the second subset of wells.

5. The method of claim 4, wherein
the array of wells is arranged in parallel rows of wells; and
closing the first subset of wells while opening the second subset of wells is performed sequentially on a row-by-row basis across the array of wells.

6. The method of claim 4 further comprising energizing opposing electromagnetic coils surrounding respective attractor posts formed in an opposing substrate positioned opposite and above the substrate to attract and hold MNPs released from covering the second subset of wells.

7. The method of claim 6, wherein
a first subset of the attractor posts and corresponding opposing electromagnetic coils corresponds to the first subset of wells; and
closing the first subset of wells further comprises contemporaneously deenergizing the opposing magnetic coils around the first subset of the attractor posts to release MNPs to cover the first subset of wells.

8. The method of claim 1 further comprising flowing a washing solution to wash unreceived nucleotides of the specified nucleobase type away from the second subset of wells and off the substrate.

9. The method of claim 2 further comprising
flowing a de-blocking solution over the substrate to remove the chemical blocker from the received and bound nucleotides of the specified nucleobase type in the second subset of wells; and
flowing a washing solution over the substrate to wash remaining de-blocking solution out of the second subset of wells and off the substrate.

10. The method of claim 1 further comprising repeating closing, flowing, receiving, and binding of claim 1 with respect to nucleotides of another nucleobase type.

11. A control system for serial and contemporaneous synthesis of disparate deoxy-ribonucleic acid (DNA) or ribonucleic acid (RNA) strands in an array of wells defined within a substrate comprising
a controller including a processor and a memory, the memory further including a control application and a data structure stored thereon, and wherein the control application is executable by the processor to
designate a first subset of wells in the array of wells defined within the substrate and containing a precursor nucleotide chain as proscribed from receiving a nucleotide of a specified nucleobase type for a synthesis cycle, wherein the designations is based, at least in part, upon sequence instruction data stored in the data structure;
close the first subset of wells;
flow a solution of a binding reaction enzyme and nucleotides of the specified nucleobase type bound with a corresponding chemical blocker over the array of wells for receipt in each of a second subset of wells of the array of wells that are open as designated by the sequence instruction data to receive the nucleotide of the specified nucleobase type for binding of the received nucleotides of the specified nucleobase type with assistance of the binding reaction enzyme to corresponding precursor nucleotide chains in the second subset of wells.

12. The control system of claim 11, wherein upon execution, the control application further directs the processor to energize electromagnetic coils defined respectively around each well of the first subset of wells to attract corresponding magnetic nanopore particles (MNPs) to cover the first subset of wells.

13. The control system of claim 11, wherein upon execution, the control application further directs the processor to open the second subset of wells by deenergizing electromagnetic coils defined respectively around each well of the second subset of wells to release respective magnetic nanopore particles (MNPs) covering the second subset of wells.

14. The control system of claim 12, wherein upon execution, the control application further directs the processor to open the second subset of wells by deenergizing electromagnetic coils defined respectively around the each well of the second subset of wells to release respective MNPs covering the second subset of wells.

15. The control system of claim 14, wherein
the array of wells is arranged in parallel rows of wells; and
the control application further directs the processor to close the first subset of wells while opening the second subset of wells sequentially on a row-by-row basis across the array of wells.

16. The control system of claim 14, wherein upon execution, the control application further directs the processor to energize opposing electromagnetic coils surrounding respective attractor posts formed in an opposing substrate positioned opposite and above the substrate to attract and hold MNPs released from covering the second subset of wells.

17. The control system of claim 16, wherein
a first subset of the attractor posts and corresponding opposing electromagnetic coils corresponds to the first subset of wells; and
the control application further directs the processor to close the first subset of wells by contemporaneously deenergizing the opposing magnetic coils around the first subset of the attractor posts to release MNPs to cover the first subset of wells.

18. The control system of claim 11, wherein upon execution, the control application further directs the processor to flow a washing solution to wash unreceived nucleotides of the specified nucleobase type away from the second subset of wells and off the substrate.

19. The control system of claim 12, wherein upon execution, the control application further directs the processor to
flow a de-blocking solution over the substrate to remove the chemical blocker from the received and bound nucleotides of the specified nucleobase type in the second subset of wells; and
flow a washing solution over the substrate to wash remaining de-blocking solution out of the second subset of wells and off the substrate.

20. The control system of claim 12, wherein upon execution, the control application further directs the processor to repeat the designating, closing, and flowing of claim 1 for binding nucleotides of another nucleobase type.
